(19) United States
(12) Patent Application Publication
(10) Pub. No.: US 2014/0286991 A1
(43) Pub. Date: Sep. 25, 2014

(54) COMBINED CELL BASED GP96-IG-SIV/HIV, RECOMBINANT GP120 PROTEIN VACCINATION FOR PROTECTION FROM SIV/HIV

(71) Applicants: Eckhard R. Podack, Miami, FL (US); Natasa Strbo, Miami, FL (US); Genoveffa Franchini, Washington, DC (US); Monica Vaccari, Washington, DC (US)

(72) Inventors: Eckhard R. Podack, Miami, FL (US); Natasa Strbo, Miami, FL (US); Genoveffa Franchini, Washington, DC (US); Monica Vaccari, Washington, DC (US)

(73) Assignee: UNIVERSITY OF MIAMI, MIAMI, FL (US)

(21) Appl. No.: 14/001,112

(22) PCT Filed: Feb. 23, 2012
(86) PCT No.: PCT/US2012/026256

§ 371 Date: Mar. 10, 2014

Related U.S. Application Data
(60) Provisional application No. 61/445,884, filed on Feb. 23, 2011.

(51) Int. Cl. A61K 39/21; A61K 35/14

(52) U.S. Cl. CPC A61K 39/21; A61K 35/17; A61K 2039/5158
USPC 424/207.1

(57) ABSTRACT

Compositions are provided comprising heat shock protein, immunoglobulins and retroviral antigens to induce systemic and mucosal immunity to infection from retroviruses such as Human Immunodeficiency Virus (HIV). Methods of treatment provided comprise administration of the compositions, which boost the immune systems response to the retroviral antigens or immunogens.

### CROSS-REFERENCE TO RELATED APPLICATIONS

The present application claims the priority of U.S. provisional patent application No. 61/445,884 entitled “COMBINED CELL BASED GP96-IG-SIV/HIV, RECOMBINANT GP120 PROTEIN VACCINATION FOR PROTECTION FROM SIV/HIV” filed Feb. 23, 2011, which is incorporated herein by reference in its entirety.

### STATEMENT REGARDING FEDERALLY SPONSORED RESEARCH

This invention was made with U.S. government support under grant number R33AI073234 which was awarded by the National Institutes of Health. The U.S. government may have certain rights in the invention.

### FIELD OF THE INVENTION

Embodiments of the invention comprise compositions of cell secreted adjuvant and antigen carrier, antigens and methods of use. Further embodiments are directed to live cells for producing vaccines over periods of time.

### BACKGROUND

Worldwide, heterosexual activity accounts for three-quarters of all HIV infections. In Europe and the USA, high-risk groups are homosexual and bisexual men, prostitutes, intravenous drug users sharing needles, and hemophiliacs and other patients treated with contaminated blood products. The different distribution of risk in developed and developing countries occurs because different types of the virus are more common in different regions. The virus has a short life outside the body, which makes transmission of the infection by methods other than sexual contact, blood transfusion, and shared syringes extremely unlikely. Pregnant women infected with HIV are unlikely to pass it on to the fetus while in the uterus, but are quite likely to do so via vaginal fluids during birth or after breast-feeding the child. More than 90% of children born to an HIV-positive mother will contract the disease unless their parent has been treated with antiretroviral drugs.

In 1991, the World Health Organization (WHO) spearheaded a vaccine development etlott centered on Brazil, Thailand, and Uganda. With the creation of UNAIDS in 1996, the program was taken over by the UN organization. Trials of an HIV vaccine began in he USA in June 1998 with 5,000 high-risk volunteers (gay men and those with HIV-infected partners) receiving the vaccine or a placebo.

In February 2003, US biotechnology company VaxGen announced that their 3-year study of a vaccine on a group of 5,000 volunteers had resulted in only a limited success. Overall, their vaccine was only able to reduce the rate of HIV infection by 3.8%. However there was some hope of a partial use for the drug, as it was able to reduce the infection rate among black and Asian volunteers by 67%. VaxGen tested their vaccine AIDVAX in Thailand in November 2003, a program involving 2,500 drug users, but this trial was a failure with the vaccine being unable to prevent the spread or progress of the disease.

### SUMMARY

This Summary is provided to present a summary of the invention to briefly indicate the nature and substance of the invention. It is submitted with the understanding that it will not be used to interpret or limit the scope or meaning of the claims.

Embodiments are directed to a combination composition or vaccine comprising gp96-Ig as an adjuvant and antigen carrier for CD8⁺ cytotoxic T lymphocyte (CTL) generation and as an adjuvant for gp120 specific antibody production (Th1 antibodies). The antigen in the composition comprises HIV or SIV antigens, for example, capsid antigens, glycoproteins, envelope antigens, nuclear antigens, or combinations thereof. The combination composition in some embodiments comprises isolated cells which release gp96-Ig over a period of time. The cells can be from any source, including donor derived, cell-lines and the like.

Other aspects are described infra.

### BRIEF DESCRIPTION OF THE DRAWINGS

FIGS. 1A-1D show that the Gp96SIV-Ig vaccines induce cellular and humoral immune responses. FIG. 1A: Schematics of the vaccination and challenge protocol. FIG. 1B: Polyepitope specific rectal lamina propria CD8⁺ T cells secrete TNFα, IFNγ upon SIV-specific peptide stimulation. SIV-specific CD8 T cell responses at week 26 were detected using pools of 15-meric peptides overlapping by 11 amino acids covering the entire Gag, Nef, and Env proteins by multiparameter TNFα and IFNγ ICS assay. Intracellular staining for TNFα, IFNγ was performed on freshly isolated rectal lamina propria mononuclear cells stimulated for 5 h with overlapping SIV peptides in the presence of monensin and brefeldin A. After gating on live, CD3⁺ CD8⁺ T cells, frequency of cytokine positive cells was determined. FIG. 1C: SIVmac251 Env ELISA at week 5 and 26 (FIG. 1D) SIV_{mac251} Env specific antibody secreting cells (ASC) at week 26 was determined by ELISPOT. Error bars represent s.e.m.

FIGS. 2A-2D show the protective efficacy of the gp96SIV-1 g vaccines. FIG. 2A: Mean SIV RNA copies per ml plasma are depicted for each vaccine group at weeks 3, 7, 15 and 17 post challenge and (FIG. 2B) SIV RNA copies per ml plasma for individual monkey. FIG. 2C: Number of challenges required for acquisition of infection in each vaccine group. FIG. 2D: Statistical analyses include the number of challenges required for 50% infection, hazard ratios with 95% confidence intervals (CI) and per-exposure vaccine protection in each group. P-values reflect Wald test using a proportional hazard model

FIGS. 3A-3C show the correlates of protection against acquisition of infection with the gp96SIV-Ig vaccines. Correlation of mean OD for the SIV_{mac251} Env antibody (FIG. 3A), frequency of SIV_{mac251} Env-specific antibody secreting cells (FIG. 3B) and plasmablasts (FIG. 3C) in the blood at week 26 with the number of challenges required to establish infection. Correlates analyses included 12 gp96SIV-Ig+gp120 vaccinated monkeys. P values reflect Spearman rank-correlation tests.

FIG. 4 shows the MHC type I, TRIM5α expression (R-restrictive TRIM5α polymorphism) and gender (F-female; M-male) of 36 Rhesus macaques.

FIG. 5 shows the protective efficacy of the gp96SIV-Ig vaccines. Number of challenges required for acquisition of infection in vaccine group with (black line) or without (gray line) animals with TRIM5α restrictive alleles.

FIGS. 6A, 6B show the protective efficacy of the gp96SIV-Ig vaccines. Number of challenges required for acquisition of infection in each vaccine group (FIG. 6A), infection times were scored when the animal had positive virus titers (titers were assessed 5 days after each challenge). FIG. 6B: Infection times were rescored in the revised failure time file with failures recorded 1 challenge earlier (for those animals with viral loads >10⁶ RNA copies/1 ml plasma on the day of first positive detection).

FIG. 7 shows that the Gp96SIV-Ig vaccines induce cellular immune responses in gut mucosa. Polyepitope specific rectal lamina propria CD8⁺ T cells express IL-2 and CD107a, upon SIV-specific peptide stimulation. SIV-specific CD8 T cell responses at week 26 were detected using pools of 15-meric peptides overlapping by 11 amino acids covering the entire Gag, Nef, and Env proteins by multiparameter ICS assay. Intracellular staining for IL-2, CD107a was performed on freshly isolated rectal lamina propria mononucler cells stimulated for 5 h with overlapping SIV peptides in the presence of monensin and brefeldin A. After gating on live, CD3⁺ CD8⁺ T cells, frequency of cytokine or CD107a positive cells was determined.

FIGS. 8A to 8D show that Gp96-SIV immunization induces SIV-gag and SIV-tat-specific CD8+ T cells in lamina propria and intraepithelial compartment of rectal mucosa. Total of eight Mamu-A01+ Rhesus macaques were immunized with gp96-SIV, gp96-SIV+ recombinant gp120 or gp96-Mock by intraperitoneal route with cells secreting 10 mg of gp96-Ig within 24 h. Immunization was administered 3 times at weeks 0, 6 and 25. Samples were harvested from rectal mucosa at week 7 and week 26 (5 days after 2nd and 3rd vaccination). FIGS. 8A and 8B: SIV-Gag-CD8 T cells were detected by Mamu-A*01/Gag181-189 CM9 (CTPYDINQM (SEQ ID NO: 1); Gag-CM9) and Tat 28-35 SL8 (TTPESANL (SEQ ID NO: 2); Tat-SL8) tetramer staining. After gating on the CD8⁺ population, the percentage of tetramer-positive cells was determined. FIGS. 8C and 8D: Phenotype analysis of CD8⁺ SIV-gag⁺ T cells in lamina propria and intraepithelial compartment. The markers CD28 and CD95 define the central memory (T_{CM}), transitional memory (T_{TM}) and effector memory (T_{EM}) among rhesus macaque T cells. T_{CM}, T_{TM} and T_{EM} cells expressing CD28⁺CD95⁺, CD28⁺CD95⁻ and CD28⁻CD95⁻ phenotypes, respectively.

FIGS. 9A and 9B show the short lived plasmablast induction by gp96SIV-Ig+gp120 vaccination. Short lived plasmablast cells (SPBs) were measured in blood by flow cytometry 5 days after the last vaccination (week 26). FIG. 9A: Shown is the gating strategy and frequency of the SPBs (CD3^{neg}/CD20^{neg}/CD21^{low}/CD27^{hi}/Ki67⁺) for a representative gp96SIV+gp120 and gp96Mock vaccinated animals. FIG. 9B: Mean frequencies of SPBs±standard error of the mean is reported for 11 mock macaques and for 12 vaccinated macaques.

FIG. 10 shows the SIV Env specific IgA antibody responses. The top panel shows SIVmac251 Env specific IgA ELISA at week 5 and 26. The lower two graphs show the total and Env specific IgA antibody secreting cells (ASCs) were quantified by ELISPOT. Peripheral blood mononuclear cells (PBMCs) collected from gp96SIV+gp120 and Mock vaccinated monkeys were assayed for total and SIV gp120 specific IgA ASCs by ELISPOT assay at 5 days after 3rd vaccination. Multiscreen 96-well plate were coated either with goat-anti-monkey IgG or recombinant SIV gp120. PBMCs were added (2×10⁴/well for total IgA or 2×10⁵ for Env-specific IgA) and incubated overnight at 37° C. Following blocking, plates were incubated with biotinylated goat anti-monkey IgA. After addition of HRP-avidin conjugate, plates were developed using AEC. Spots were counted using automated ELISPOT reader (C.T.L. ImmunoSpot 5.0.3). Mean values (number of spots per million of PBMCs)±standard error of the mean is reported for 12 mock macaques and for 12 vaccinated macaques.

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homo logs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the molecules disclosed herein, gp96-Ig is not limited to a single species but the human immunoglobulin is preferred, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### DEFINITIONS

The terminology used herein is for the purpose of describing particular embodiments only and is not intended to be limiting of the invention. As used herein, the singular forms “a”, “an” and “the” are intended to include the plural forms as well, unless the context dearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably “within 2-fold, of a value. Where particular values are described in the application and claims, unless otherwise stated the term “about” meaning within an acceptable error range for the particular value should be assumed.

The term “heat shock protein”, as used herein, refers to any protein which exhibits increased expression in a cell when the cell is subjected to a stress. It is to be understood that the term “heat shock protein” encompasses both proteins that are induced in response to stress conditions and homologs of such proteins that are constitutively expressed. In preferred non-limiting embodiments, the heat shock protein is originally derived from a eukaryotic cell; in more preferred embodiments, the heat shock protein is originally derived from a mammalian cell. In preferred embodiments, the heat shock protein is human. For example, but not by way of limitation, heat shock proteins which may be used according to the invention include BiP (also referred to as grp78), hsp/hsc70, gp96 (grp94), hsp60, hsp40, and hsp90. Preferred heat shock proteins are BiP, gp96, and hsp70. In preferred embodiments, the heat shock protein is gp96. Naturally occurring or recombinantly derived mutants of heat shock proteins may also be used according to the invention.

An “immunogen” or “antigen” is a compound or molecule derived from the cell or organism that elicits in a subject an antibody-mediated immune response (i.e., a “B cell” response or humoral immunity), a cell-mediated immune response (i.e. a “T cell” response), or a combination thereof. A compound or molecule may be composed of amino acids, carbohydrates, nucleic acids or lipids individually or in any combination. A cell-mediated response can involve the mobilization helper T cells, cytotoxic T-lymphocytes (CTLs), or both. Preferably, an immunogenic polypeptide elicits one or more of an antibody-mediated response, a CD4⁺ Th1 mediated response (Th1: type 1 helper T cell), and a CD8⁺ T cell response. It should be understood that the term “polypeptide” as used herein refers to a polymer of amino acids and does not refer to a specific length of a polymer of amino acids. Thus, for example, the terms peptide, oligopeptide, and protein are included within the definition of polypeptide. As used herein, “contacting” means placing the biological sample in sufficient proximity to the agent and under the appropriate conditions of, e.g., concentration, temperature, time, ionic strength, to allow the specific interaction between, for example, an agent and nucleic acid or polypeptide that are present in the biological sample. In general, the conditions for contacting the agent with the biological sample are conditions known by those of ordinary skill in the art to facilitate a specific interaction between a molecule and its cognate (e.g., a protein and its receptor cognate, an antibody and its protein antigen cognate, a nucleic acid and its complementary sequence cognate) in a biological sample. Exemplary conditions for facilitating a specific interaction between a molecule and its cognate are described in U.S. Pat. No. 5,108,921, issued to Low et al.

“Treating” or “treatment” of a state, disorder or condition includes: (1) Preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or (2) Inhibiting the state, disorder or condition, i.e., arresting, reducing or delaying the development of the disease or a relapse thereof (in case of maintenance treatment) or at least one clinical or sub-clinical symptom thereof; or (3) Relieving the disease, i.e., causing regression of the state, disorder or condition or at least one of its clinical or sub-clinical symptoms. The benefit to a subject to be treated is either statistically significant or at least perceptible to the patient or to the physician.

“Patient” or “subject” refers to mammals and includes human and veterinary subjects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount and prevents or is protective against the disease or infection.

### Combination Vaccine or Composition

Protective vaccination against many life threatening viral infections has been successful in breaking epidemics and even eliminating the disease. Vaccine induced protection against infection by HIV or highly pathogenic and virulent SIV-strains, however, has been elusive.

In general embodiments, the molecular combination for a preventative and effective vaccine to protect or treat subjects comprises an adjuvant or antigen carrier and two or more specific antigens to induce a protective or therapeutic immune response. The antigens are preferably two different antigens, for example HIV gp120 and gp160. The success of the molecular composition in vivo can be monitored via any assay, including immunoassays, viral load assays, measuring specific retroviral antigens, such as p24, etc.

Briefly, the data show that a novel, unconventional vaccine approach significantly protects macaques from mucosal infection by the highly pathogenic strain of SIV_{mac251}. Macaques were vaccinated with live, irradiated vaccine cells secreting the modified ER chaperone gp96SIVIg which is loaded with SIV antigen derived client peptides. Gp96 is a danger associated molecular pattern (DAMP) cross priming SIV-antigen specific CTL and activating antibody responses when combined with SIV envelop recombinant gp120-protein. In vivo secretion of gp96SIV for several days generates potent signals for SIV specific CTL expansion and antibody production, both of which were required for protection from SIV acquisition. Vaccination protected 3 of 12 macaques from infection by six mucosal SIV challenges and significantly delayed infection in all macaques immunized with the vaccine combination. After 7 challenges the hazard ratio was 0.27 corresponding to a 73% reduced risk of viral acquisition. Cell based, secreted gp96-vaccines, also used as therapeutic vaccines in cancer patients, have great promise to play a significant role in medicine.

In a preferred embodiment, a molecular combination composition comprises a cell secreted gp96-Ig as adjuvant and antigen carrier for CD8 CTL generation and as adjuvant for gp120 specific antibody production (Th1 antibodies). In one embodiment, live cells are utilized as a vaccine to secrete gp96-Ig over several days.

In one preferred embodiment, the composition is a combination of cell based, for example, an isolated cell-gp96-Ig-HIV-(or SIV)-gag, retanef (Rev-Tat-Nef), gp160 vaccination with recombinant gp120-protein, using gp96-Ig as adjuvant.

In another preferred embodiment, an isolated cell comprises a vector or polynucleotide encoding a combination molecule comprising at least one adjuvant and antigen carrier (e.g. gp96-Ig) and antigen to generate an immune response. The antigen comprises retroviral antigens, such as HIV or SIV antigens, mutants, variants or fragments thereof. Preferably the cell is a live cell so as to secrete the gp96-Ig over a period of time. The cells can be irradiated to prevent replication. In some embodiments, the cells are human leukocyte antigen (HLA)-matched, autologous, cell lines, or combinations thereof.

In another preferred embodiment, the adjuvant and antigen carrier can be any molecule that can serve these purposes. For example, gp96 is a 96-kDa glycoprotein of the endoplasmic reticulum that is involved in antigen processing as an intermediate carrier of peptides for presentation by major histocompatibility complex (MHC) class I molecules. This function means that gp96 carries a large array of different peptides that represent the antigenicity of the cell and can serve all MHC class I molecules. Thus the, gp96 “carries” the HIV antigen, (e.g. gp 160, gp120 combination) and generates CD8 cytotoxic T lymphocytes (CTL). The retroviral antigens can be recombinant, native or combinations thereof. Examples of retroviral antigens comprise HIV, SIV antigens such as for example, gp120, gp 160, gag, pol, env, retanef, etc. The antigens can be from any HIV or SIV variant.

In a preferred embodiment, a composition for generating viral antigen specific immune responses in vitro or in vivo, comprises a vaccine molecule having a first domain comprising at least one adjuvant or antigen carrier wherein the adjuvant or antigen carrier is a heat shock protein and an immunoglobulin, or nucleic acids encoding the heat shock protein and immunoglobulin, and, a second domain comprising at least one viral molecule.

In preferred embodiments, the heat shock protein, or nucleic acids thereof, comprise: BiP (grp78), hsp/hsc70, gp96 (grp94), hsp60, hsp40, hsp90, mutants, fragments, variants or substituted molecules thereof. Preferably, the heat shock protein or nucleic acids thereof is gp96.

In other embodiments, the immunoglobulin, or nucleic acids thereof, comprise IgG, IgA, IgD, IgM, IgE, or fragments thereof. In some embodiments, the fragments of the immunoglobulin comprise: Fc, Fab, F(ab′)₂, V_{H}, V_{L}, C_{L}, C_{H1}, C_{H2}, C_{H3}, C_{H4}, or combinations thereof.

In other embodiments, the heat shock protein and immunoglobulin are attached via covalent or non-covalent bonds, fused or linked.

In another preferred embodiment, the composition comprises at least one viral molecule comprises retroviral molecules. Preferably, the retrovirus is Human or Simian Immunodeficiency Virus (HIV, SIV). In preferred embodiments, the retroviral molecule comprises: Gag, Tat, Rev, Nef, and gp160 or fragments thereof.

In another preferred embodiment, the composition comprises a second viral molecule, wherein the second viral molecule is not associated with the vaccine molecule and is a retroviral immunogen. As used herein “not associated with the vaccine molecule” is meant to include any other immunogen which may be administered either separately or in the form of a another second vector encoding the immunogen. In this case this second vector can be comprised within the same cell as the first vector encoding the composition or can be transfected into another cell. In addition, the immunogen can also be administered as any type of molecule, e.g. recombinant gp120 peptides. In one embodiment, the second viral immunogen is a glycoprotein or nucleic acids thereof.

In another preferred embodiment, an isolated cell comprises a vector expressing a vaccine molecule having a first domain comprising at least one adjuvant or antigen carrier wherein the adjuvant or antigen carrier is a heat shock protein and an immunoglobulin, or nucleic acids encoding the heat shock protein and immunoglobulin, and, a second domain comprising at least one viral molecule. In a preferred embodiment, the heat shock protein is modified to be secreted from the host cell. In one embodiment, the cell is a human or primate cell. Preferably, the viral molecule is a retroviral molecule comprising: Gag, Tat, Rev, Nef, and gp160 or fragments thereof.

In some embodiments, the isolated cell comprises a vector expressing a second viral molecule wherein the second viral molecule is a retroviral immunogen. Examples include, without limitations: glycoproteins, envelope antigens, capsid antigens, nuclear antigens, or combinations thereof. In preferred embodiments, the heat shock protein, or nucleic acids thereof, comprise: BiP (grp78), hsp/hsc70, gp96 (grp94), hsp60, hsp40, hsp90, mutants, fragments, variants or substituted molecules thereof. Preferably, the heat shock protein or nucleic acids thereof is gp96. In embodiments, the gp96 lacks a functional endoplasmic reticulum retention sequence so that the heat shock protein-immunoglobulin is secreted. In some embodiments, the cell is irradiated.

In other embodiments, the isolated cell is a patient's autologous cell. In other embodiments, the cell is syngeneic, xenogeneic, allogeneic

In another embodiment, a vaccine comprises a plurality of cells expressing an adjuvant comprising a heat shock protein (hsp) and immunoglobulin (Ig) and, one or more retroviral molecules wherein the one or more retroviral molecules comprise Gag, Tat, Rev, Nef, and gp160 or fragments thereof. Preferably, the cells secrete the adjuvant. In another embodiment, the vaccine further comprises a retroviral immunogen wherein the immunogen comprises glycoproteins, envelope antigens, capsid antigens, nuclear antigens, or combinations thereof. Preferably a second immunogen is also administered comprising retroviral immunogens, e.g. recombinant gp120.

In other embodiments, a method of inducing an antigen-specific immune response against a virus in a subject, the method comprises administering to the subject an adjuvant composition comprising a host cell expressing a secretable vaccine molecule having a first domain comprising at least one adjuvant or antigen carrier wherein the adjuvant or antigen carrier is a heat shock protein and an immunoglobulin, and, a second domain comprising at least one viral molecule; administering a viral immunogen whereby the immunogen induces an antigen-specific immune response against a virus in a subject. Preferably, the heat shock protein, comprises: BiP (grp78), hsp/hsc70, gp96 (grp94), hsp60, hsp40, hsp90, mutants, fragments, variants or substituted molecules thereof. In a preferred embodiment, the heat shock protein is a gp96 lacking a functional endoplasmic reticulum retention sequence. In preferred embodiments, the viral molecule of the second domain is a retroviral molecule comprising: Gag, Tat, Rev, Nef, and gp160 or fragments thereof.

In preferred embodiments, administering the composition results in expansion of T cells specific for the antigen in the subject's peripheral blood and the subjects mucosa. In other preferred embodiments, B cells are also expanded producing antigen specific antibodies.

In another preferred embodiment, a method of preventing or treating a retroviral infection in a subject comprises administering a therapeutically effective amount of the compositions embodied herein, comprising: a host cell expressing a vaccine molecule having a first domain comprising at least one adjuvant or antigen carrier wherein the adjuvant or antigen carrier is a heat shock protein and an immunoglobulin (hsp-Ig), and, a second domain comprising at least one viral molecule. In preferred embodiments, a viral immunogen is administered to the subject. The administration of the viral immunogen can be co-administered with the hsp-1 g, prior to or after the hsp-1 g. In other embodiments, the immunogen is administered in doses over periods of time. The hsp-Ig can be administered as a cell composition which secretes the composition over periods of time.

In further embodiments, an immunogen may be associated with an infectious disease, and, as such, may be a bacterium, virus, protozoan, mycoplasma, fungus, yeast, parasite, or prion. For example, but not by way of limitation, the immunogen may be a human papilloma virus, a herpes virus such as herpes simplex or herpes zoster, a retrovirus such as human immunodeficiency virus 1 or 2, a hepatitis virus, an influenza virus, a rhinovirus, respiratory syncytial virus, cytomegalovirus, adenovirus, Mycoplasma pneumoniae, a bacterium of the genus Salmonella, Staphylococcus, Streptococcus, Enterococcus, Clostridium, Escherichia, Klebsiella, Vibrio, Mycobacterium, amoeba, a malarial parasite, Trypanosoma cruzi, etc. Preferably, the immunogen is a retroviral antigen.

In one preferred embodiment, the immunoglobulin (Ig) or fragment thereof, comprises IgA, IgM, IgG, IgE, or IgD.

In one embodiment, a method of treating a patient suffering from a virus infection comprises administering to a patient an effective amount of a heat shock protein—immunoglobulin composition, e.g. gp96-Ig, and an immunogen wherein the immunogen comprises viral antigenic epitopes, for example, HIV antigenic epitopes. In other preferred embodiments, autologous cells are cultured ex vivo with the compositions herein and re-infused into a patient. Desired antigens are administered to the cultures and cells are re-infused into a patient once a desired amount of antigen specific T cells have been generated.

In a preferred embodiment, a method of inducing both mucosal and systemic and systemic immunity comprising administering to a patient in need thereof, a therapeutically effective amount of a vaccine having at least one heat shock protein, at least one immunogen from one or more pathogens or diseases, fragments variants, derivatives, mutants, or combinations thereof.

In preferred embodiments, a method of inducing HIV/SIV antigen specific mucosal and systemic immunity and systemic immunity in vivo, comprises administering to a patient in need thereof, a therapeutically effective amount of antigen comprising a heat shock protein, or fragments thereof, such as for example, gp96. Preferably the gp96 is secreted (gp96-Ig). The heat shock protein is not just limited to gp96 but extends to all other heat shock proteins. Heat shock proteins are among the most highly conserved proteins in existence. For example, DnaK, the hsp70 from E. coli has about 50% amino acid sequence identity with hsp70 proteins from excoriates (Bardwell, et al., 1984, Proc. Natl. Acad. Sci. 81:848-852). The hsp60 and hsp90 families also show similarly high levels of intrafamily conservation (Hickey, et al., 1989, Mol. Cell. Biol. 9:2615-2626; Jindal, 1989, Mol. Cell. Biol. 9:2279-2283). In addition, the hsp60, hsp70 and hsp90 families are composed of proteins that are related to the stress proteins in sequence, for example, having greater than 35% amino acid identity, but whose expression levels are not altered by stress. Therefore it is contemplated that the definition of heat shock protein or stress protein, as used herein, embraces other proteins, muteins, analogs, and variants thereof having at least 35% to 55%, preferably 55% to 75%, and most preferably 75% to 85% amino acid identity with members of the three families whose expression levels in a cell are enhanced in response to a stressful stimulus.

In another preferred embodiment, administration of the gp96-Ig composition to a patient in need thereof induces an HIV/SIV antigen specific mucosal and systemic immune response comprising induction of an antigen specific T cell immune response. Preferably, the antigen specific T cell response is polyspecific comprising CD8, CD4 T cells, innate dendritic cell, natural killer cells (NK), and memory CD8⁺ T cells.

In another preferred embodiment, the HIV/SIV antigen comprises: an isolated cell having a plasmid encoding gp96-Ig, HIV/SIV antigens retanef (Rev-Tat-Nef), gag, gp160, fragments, variants, mutants, derivatives or combinations thereof. The retanef preferably comprises at least one of Rev, Tat, Nef, fragments, variants, mutants, derivatives or combinations thereof.

In another preferred embodiment, the isolated cell expresses endogenous, membrane bound, secreted or combinations thereof, of at least one of the molecules comprising: gp96, retanef, gag, gp160, fragments, variants, mutants, derivatives or combinations thereof.

In another preferred embodiment, the isolated cells comprise autologous, syngeneic, heterologous, xenogeneic cells, cell lines, or combinations thereof.

In another preferred embodiment, a method of preventing HIV in a patient at risk of being infected with HIV, or treating a patient, infected with HIV, comprises administering to the patient in need thereof, a therapeutically effective amount of antigen comprising gp96, wherein the antigen induces an HIV/SIV antigen specific mucosal and systemic immunity comprising an antigen specific B cell and T cell immune response.

In another preferred embodiment, the HIV/SIV antigen induces immune cells comprising central memory T cells (T_{CM}; CD95⁺ CD28⁺), effector memory T cells (T_{EM}; CD95⁺CD28⁻) or naive T cells (CD95^{low} CD28^{int}).

In another preferred embodiment, an isolated nucleic acid encoding at least one molecule comprising: gp96-Ig, HIV/SIV antigens retanef (Rev-Tat-Nef), gag, gp 160, fragments, variants, mutants, derivatives or combinations thereof.

In another preferred embodiment, an isolated nucleic acid encoding at least one molecule comprising: gp96-Ig, an immunogen (e.g. tumor antigen, antigens associated with infectious organisms, etc.), fragments, variants, mutants, derivatives or combinations thereof.

In another preferred embodiment, the expression vector is a bicistronic vector. In one aspect, the vector comprises an SV40 promoter, however, any type of promoter that is functional in different cell types can be used, including tissue specific promoters. Examples of promoters useful to practice the present invention, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein.

In one preferred embodiment, the encoded molecules are endogenous, membrane bound, secreted or combinations thereof. Preferably, the molecules are secreted.

In another preferred embodiment, a fusion protein comprising at least one of: gp96-Ig, HIV/SIV antigens retanef (Rev-Tat-Nef), gag, gp160, fragments, variants, mutants, derivatives or combinations thereof.

In another preferred embodiment, a fusion protein comprising at least one of: gp96-Ig, immunogen, fragments, variants, mutants, derivatives or combinations thereof. Preferably the gp96 is fused or linked to the immunogen and the molecule is secreted. The molecule can be encoded by an expression vector in a cell, preferably a mammalian cell. The cell can be obtained from a patient, which is cultured ex-vivo; the cell is contacted with the expression vector; cells producing the molecule are then re-infused into the patient, via any mode, such as i.v. i.p. etc.

In another preferred embodiment, an isolated cell comprising a nucleic acid molecule encoding at least one or more of: gp96-Ig, HIV/SIV antigens retanef (Rev-Tat-Nef), gag, gp160, fragments, variants, mutants, derivatives or combinations thereof.

In another preferred embodiment, a method of inducing Human Immunodeficiency Virus (HIV) specific immune response in vivo or in vitro, comprising: administering to the patient in need thereof, a therapeutically effective amount of an HIV/SIV specific molecule (for example, HIV/SIV antigens retanef (Rev-Tat-Nef), gag, gp160, fragments, variants, mutants, derivatives or combinations thereof) and an adjuvant or antigen carrier comprising gp96-Ig. Preferably, the immunogen comprising gp96 induces an HIV/SIV antigen specific mucosal and systemic immunity comprising an antigen specific T and B cell immune response. The antigen specific T cell response is preferably, polyspecific comprising CD8⁺ and CD4⁺ T cells, wherein the T cells co-express and produce IFNγ and IL-2. In another embodiment, the HIV/SIV antigen specific mucosal and systemic immunity further comprises innate dendritic cell, natural killer cells (NK), CD103⁺ cells, CD8⁺ CD103⁺ T cells, and/or memory CD8⁺ T cells. Preferably, the memory cells comprise central memory T cells (T_{CM}; CD95⁺CD28⁺), effector memory T cells (T^{EM}; CD95⁺CD28⁻) or naive T cells (CD95^{low} CD28⁺).

As discussed above, the heat shock protein can be from any family of hsp and the immunogen is selected from the disease of interest. The illustrative examples herein described the retanef, gag for HIV. The compositions comprising hsp can be fused, linked, covalently or noncovalently bound to the antigenic molecules or immunogens and are administered to elicit an effective specific immune response to the molecules. In accordance with the methods described herein, the hsp-antigenic molecule complexes are preferably purified in the range of 60 to 100 percent of the total mg protein, or at least 70%, 80% or 90% of the total mg protein. In another embodiment, the hsp-antigenic molecule complexes are purified to apparent homogeneity, as assayed by sodium dodecyl sulfate-polyacrylamide gel electrophoresis.

In a preferred embodiment, the complexes of hsp70, hsp90 and gp96 with peptides are prepared and purified postoperatively from, for example, tumor cells obtained from the cancer patient or cells from an infected patient, such as for example, an HIV infection.

In accordance with the methods described herein, immunogenic or antigenic peptides that are endogenously complexed to hsps or MHC antigens can be used as antigenic molecules. For example, such peptides may be prepared that stimulate cytotoxic T cell responses against different tumor antigens (e.g., tyrosinase, gp100, melan-A, gp75, mucins, etc.) and viral proteins including, but not limited to, proteins of immunodeficiency virus type I (HIV-I), human immunodeficiency virus type H (HIV-II), hepatitis type A, hepatitis type B, hepatitis type C, influenza, Varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus and polio virus. In the embodiment wherein the antigenic molecules are peptides linked to hsps in vivo, the complexes can be isolated from cells, or alternatively, produced in vitro from purified preparations each of hsps and antigenic molecules.

In another preferred embodiment, mucosal and systemic immune responses are modulated by administration of a composition comprising an hsp linked to one or more immunogens. The molecule is preferably a secreted molecule and can be administered either alone as an expression vector or in the context of a cell comprising the vector which encodes the desired molecule. For example, the immunogen comprises one or more antigens derived from immunogenic or antigenic peptides. For example, such peptides may be prepared that stimulate cytotoxic T cell responses against different tumor antigens (e.g., tyrosinase, gp100, melan-A, gp75, mucins, etc.) and viral proteins and/or other pathogens including, but not limited to, antigens of human immunodeficiency viruses, such as HIV-1 and HIV-2, polio viruses, hepatitis A virus, human coxsackie viruses, rhinoviruses, echoviruses, equine encephalitis viruses, rubella viruses, dengue viruses, encephalitis viruses, yellow fever viruses, coronaviruses, vesicular stomatitis viruses, rabies viruses, Ebola viruses, parainfluenza viruses, mumps virus, measles virus, respiratory syncytial virus, influenza viruses, Hantaan viruses, bunga viruses, hemorrhagic fever viruses, reoviruses, orbiviruses, rotaviruses, Hepatitis B virus, parvoviruses, papilloma viruses, polyoma viruses, adenoviruses), herpes simplex virus (HSV) 1 and 2, varicella zoster virus, cytomegalovirus (CMV), variola viruses, vaccinia viruses, pox viruses, African swine fever virus, the unclassified agent of delta hepatitis, the agents of non-A, non-B hepatitis; infectious bacteria like: Helicobacter pylori, Borrelia burgdorferi, Legionella pneumophila, Mycobacterium tuberculosis, Mycobacterium bovis (BCG), Mycobacterium avium, Mycobacterium intracellulare, Staphylococcus aureus, Neisseria gonorrhoeae, Neisseria meningitidis, Listeria monocytogenes, Streptococcus pyogenes, Streptococcus pneumoniae, Haemophilus influenzae, Moraxella catharralis, Klebsiella pneumoniae, Bacillus anthracia, Corynebacterium diphtheriae, Clostridium perfringens, Clostridium tetani, Enterobacter aerogenes, Klebsiella pneumoniae, Pasturella multocida, and Treponema pallidum; infectious fungi like: Cryptococcus neoformans, Histoplasma capsulatum, Coccidioides immitis, Blastomyces dermatitidis, Candida albicans; and infectious protists like, for example: Plasmodium falciparum, Trypanosoma cruzi, Leishmania donovani and Toxoplasma gondii; as well as infectious fungi such as those causing e.g., histoplasmosis, candidiasis, cryptococcosis, blastomycosis and cocidiodomycosis; as well as Candida spp. (i.e., C. albicans, C. parapsilosis, C. krusei, C. glabrata, C. tropicalis, or C. lusitaniaw); Torulopus spp. (i.e., T. glabrata); Aspergillus spp. (i.e., A. fumigalus), Histoplasma spp. (i.e., H. capsulatum); Cryptococcus spp. (i.e., C. neoformans); Blastomyces spp. (i.e., B. dermatilidis); Fusarium spp.; Trichophyton spp., Pseudallescheria boydii, Coccidioides immits, and Sporothrix schenckii, and; as well as human tumoral cells. In the embodiment wherein the antigenic molecules are peptides noncovalently complexed to hsps in vivo, the complexes can be isolated from cells, or alternatively, produced in vitro from purified preparations each of hsps and antigenic molecules.

Antigens or antigenic portions thereof can be selected for use as antigenic molecules, for association with hsps, from among those known in the art or determined by immunoassay to be able to bind to antibody or MHC molecules (antigenicity) or generate immune response (immunogenicity) as described above. In preferred embodiments, the vaccine stimulates the mucosal and systemic immune response and systemic immune response. This is especially important in cases such as for example, HIV whereby the point of entry is usually via mucosa. The regulation of the mucosal and systemic immune response is also important in those cases where the immune system is associated with the disease. Examples include, colitis, Crohn's disease, inflammatory bowel diseases, arthritis, autoimmune diseases or disorders, allergies, allergic reactions, asthma, lung inflammation and the like.

In other embodiments, the immunogens or antigens can be tumor antigens.

The mucosal and systemic immune system, consisting of lymphoid tissues associated with the lacrimal, salivary, gastrointestinal, respiratory and urogenital tracts and lactating breasts, quantitatively contains the majority of the lymphoid tissue of the body. There are a number of important features of the gastrointestinal mucosal and systemic immune system: the mucosal and systemic immune system contains specialized structures, such as the Peyer's patches, where immune responses are likely to be initiated; there is a pattern of relatively specific recirculation of lymphoid cells to the mucosa, known as mucosal and systemic homing; subsets of lymphoid cells, particularly IgA B cells and memory T cells, predominate at mucosal and systemic surfaces; and the predominant mucosal and systemic immunoglobulin, secretory IgA, is particularly well adapted to host defense at mucosal and systemic surfaces. These elements of the gastrointestinal mucosal and systemic immune system function together to generate an immune response which on the one hand protects the host from harmful pathogens, but on the other hand is tolerant of the ubiquitous dietary antigens and normal microbial flora.

Formulations

The invention contemplates delivery of the gp96-Ig molecules comprising; nucleic acids, polypeptides, peptides, vectors, cells comprising gp96-Ig nucleic acids or polypeptides, splice variants and the like. Delivery of polypeptides and peptides can be accomplished according to standard vaccination protocols which are well known in the art.

In another preferred embodiment, a vector comprises an hsp-immunogen such as for example, gag, retanef, gp160-gp96-Ig polynucleotide, natural splice variants, deletions, variants, mutants or active fragments thereof.

A number of vectors are known to be capable of mediating transfer of gene products to mammalian cells, as is known in the art and described herein. A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Suitable nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. Preferably, the viral vector comprises a strong eukaryotic promoter operably linked to the polynucleotide e.g., a cytomegalovirus (CMV) promoter.

Additionally preferred vectors include viral vectors, fusion proteins and chemical conjugates. Retroviral vectors include Moloney murine leukemia viruses and HIV-based viruses. One preferred HIV-based viral vector comprises at least two vectors wherein the gag and pol genes are from an HIV genome and the env gene is from another virus. DNA viral vectors are preferred. These vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.: 90 7603 (1993); Geller, A. I., et al., Proc Natl. Acad. Sci. USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11 (2):21 (1989) and Maurer, R. A., Bethesda Res. Lab. Focus, 11 (2):25 (1989).

Replication-defective recombinant adenoviral vectors can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the gp96-Ig intracellularly. See for example, Chen et al, BioTechniques, 34: 167-171 (2003), which is incorporated herein, by reference, in its entirety.

Expression of the hsp-immunogen molecules may be controlled by any promoter/enhancer element known in the art, but these regulatory elements must be functional in the host selected for expression. Promoters which may be used to control gp96-Ig gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the β-lactamase promoter (VIIIa-Kamaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., Cell 38:639-646, 1984; Ornitz et al., Cold Spring Harbor Symp. Quant. Biol. 50:399-409, 1986; MacDonald, Hepatology 7:425-515, 1987); insulin gene control region which is active in pancreatic beta cells (Hanahan, Nature 315:115-122, 1985), immunoglobulin gene control region which is active in lymphoid cells (Grosschedl et al., Cell 38:647-658, 1984; Adames et al., Nature 318:533-538, 1985; Alexander et al., Mol. Cell. Biol. 7:1436-1444, 1987), mouse mammary tumor virus control region which is active in testicular, breast, lymphoid and mast cells (Leder et al., Cell 45:485-495, 1986), albumin gene control region which is active in liver (Pinkert et al., Genes and Devel. 1:268-276, 1987), alpha-fetoprotein gene control region which is active in liver (Krumlauf et al., Mol. Cell. Biol. 5:1639-1648, 1985; Hammer et al., Science 235:53-58, 1987), alpha 1-antitrypsin gene control region which is active in the liver (Kelsey et al., Genes and Devel. 1: 161-171, 1987), beta-globin gene control region which is active in myeloid cells (Mogram et al., Nature 315:338-340, 1985; Kollias et al., Cell 46:89-94, 1986), myelin basic protein gene control region which is active in oligodendrocyte cells in the brain (Readhead et al., Cell 48:703-712, 1987), myosin light chain-2 gene control region which is active in skeletal muscle (Sani, Nature 314:283-286, 1985), and gonadotropic releasing hormone gene control region which is active in the hypothalamus (Mason et al., Science 234:1372-1378, 1986).

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67:31-40, 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2.mu. plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences; and the like.

Yeast expression systems can also be used according to the invention to express TNFR25. For example, the non-fusion pYES2 vector (XbaI, SphI, ShoI, NotI, GstXI, EcoRI, BstXI, BamHI, SacI, KpnI, and HindIII cloning sites; Invitrogen) or the fusion pYESHisA, B, C (XbaI, SphI, ShoI, NotI, BstXI, EcoRI, BamHI, SacI, KpnI, and HindIII cloning sites, N-terminal peptide purified with ProBond resin and cleaved with enterokinase; Invitrogen), to mention just two, can be employed according to the invention. A yeast two-hybrid expression system can be prepared in accordance with the invention.

One preferred delivery system is a recombinant viral vector that incorporates one or more of the polynucleotides therein, preferably about one polynucleotide. Preferably, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

In a preferred embodiment, a composition of the invention is administered to a patient via immunization routes. F or example, intra-venously, intra-muscularly, intra-peritoneally, and the like. Preferably, the immunization induces a mucosal and systemic immune response, systemic immune response.

In the case of polynucleotides or oligonucleotides, the delivery of the nucleic acid, e.g. encoding gp96-Ig, can be accomplished by ex vivo methods, i.e. by removing a cell from a subject; genetically engineering the cell to include the nucleic acid, and reintroducing the engineered cell into the subject. One example of such a procedure is outlined in U.S. Pat. No. 5,399,346. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

In another preferred embodiment, an isolated cell expresses hsp-immunogens, for example, gp96-Ig molecules. The cell can be autologous, syngeneic, xenogeneic etc, stem cell, immune cell, mucosal and systemic cell and the like.

In another embodiment, the vaccines can be administered to autologous cells, allow the cells to expand and then re-infuse the cells into the patient.

The compositions can be administered in a pharmaceutical composition, as a polynucleotide in a vector, liposomes, nucleic acids peptides and the like.

In another preferred embodiment, the compositions can be administered with one or more or additional pharmacologically active agents. As used herein, the term “pharmacologically active agent” refers to any agent, such as a drug, capable of having a physiologic effect (e.g., a therapeutic or prophylactic effect) on prokaryotic or eukaryotic cells, in vivo or in vitro, including, but without limitation, chemotherapeutics, anti-virals, toxins, radiotherapeutics, radiosensitizing agents, gene therapy vectors, antisense nucleic acid constructs or small interfering RNA, imaging agents, diagnostic agents, agents known to interact with an intracellular protein, polypeptides, and polynucleotides.

The additional pharmacologically active agent can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

The additional pharmacologically active agent need not be a therapeutic agent. For example, the agent may be cytotoxic to the local cells to which it is delivered but have an overall beneficial effect on the subject. Further, the agent may be a diagnostic agent with no direct therapeutic activity per se, such as a contrast agent for bioimaging.

In another preferred embodiment, any of the compositions embodied herein, e.g. gp160-gp96-1 g polynucleotide or peptide are labeled with a detectable marker, such as for example, fluorescent markers (e.g. GFP, RFP etc) or radiolabels.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

### Administration of Compositions

The compositions of the present invention may be administered in conjunction with one or more additional active ingredients, pharmaceutical compositions, or other vaccines. The therapeutic agents of the present invention may be administered to an animal, preferably a mammal, most preferably a human.

The pharmaceutical formulations and vaccines may be for administration by oral (solid or liquid), parenteral (intramuscular, intraperitoneal, intravenous (IV) or subcutaneous injection), transdermal (either passively or using ionophoresis or electroporation), transmucosal and systemic (nasal, vaginal, rectal, or sublingual), or inhalation routes of administration, or using bioerodible inserts and can be formulated in dosage forms appropriate for each route of administration.

The agents may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

In some embodiments, the compositions or vaccines are administered by pulmonary delivery. The composition or vaccine is delivered to the lungs of a mammal while inhaling and traverses across the lung epithelial lining to the blood stream [see, e.g., Adjei, et al. Pharmaceutical Research 1990; 7:565 569; Adjei, et al. Int. J. Pharmaceutics 1990; 63:135 144 (leuprolide acetate); Braquet, et al. J. Cardiovascular Pharmacology 1989; 13 (sup5): 143 146 (endothelin-1); Hubbard, et al. (1989) Annals of Internal Medicine, Vol. III, pp. 206 212 (α1 antitrypsin); Smith, et al. J. Clin. Invest. 1989; 84:1145-1146 (α1-proteinase); Oswein, et al. “Aerosolization of Proteins”, 1990; Proceedings of Symposium on Respiratory Drug Delivery II Keystone, Colo. (recombinant human growth hormone); Debs, et al. J. Immunol. 1988; 140:3482 3488 (interferon γ and tumor necrosis factor α); and U.S. Pat. No. 5,284,656 to Platz, et al. (granulocyte colony stimulating factor). A method and composition for pulmonary delivery of drugs for systemic effect is described in U.S. Pat. No. 5,451,569 to Wong, et al. See also U.S. Pat. No. 6,651,655 to Licalsi et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). All such devices require the use of formulations suitable for the dispensing of the therapeutic agent. Typically, each formulation is specific to the type of device employed and may involve the use of an appropriate propellant material, in addition to the usual diluents, adjuvants, surfactants and/or carriers useful in therapy. Also, the use of liposomes, microcapsules or microspheres, inclusion complexes, or other types of carriers is contemplated.

Formulations for use with a metered dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the therapeutic agent, and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Nasal or other mucosal and systemic delivery of the therapeutic agent is also contemplated. Nasal delivery allows the passage to the blood stream directly after administering the composition to the nose, without the necessity for deposition of the product in the lung. Formulations for nasal delivery include those with dextran or cyclodextran and saponin as an adjuvant.

Methods of Stimulating an Immune Response:

In a typical immunization regime employing the vaccines of the present invention, the formulations may be administered in several doses (e.g. 1-4). The dose will be determined by the immunological activity the composition produced and the condition of the patient, as well as the body weight or surface areas of the patient to be treated. The size of the dose also will be determined by the existence, nature, and extent of any adverse side effects that may accompany the administration of a particular composition in a particular patient. For example, if the compositions are cells comprising the vaccines, the number of cells will be calculated and/or the amount of, for example, gp96-Ig which is being secreted will be calculated prior to administration to a patient.

The compositions of the present invention may be administered via a non-mucosal and systemic or mucosal and systemic route. These administrations may include in vivo administration via parenteral injection (e.g. intravenous, subcutaneous, and intramuscular) or other traditional direct routes, such as buccal/sublingual, rectal, oral, nasal, topical (such as transdermal and ophthalmic), vaginal, pulmonary, intraarterial, intraperitoneal, intraocular, or intranasal routes or directly into a specific tissue. Alternatively, the compositions of the invention may be administered by any of a variety of routes such as oral, topical, subcutaneous, mucosal and systemic, intravenous, intramuscular, intranasal, sublingual, transcutaneous, subdermal, intradermal and via suppository. Administration may be accomplished simply by direct administration using a patch, needle, catheter or related device, at a single time point or at multiple time points.

Immunization via the mucosal and systemic surfaces offers numerous potential advantages over other routes of immunization. The most obvious benefits are 1) mucosal and systemic immunization does not require needles or highly-trained personnel for administration, and 2) immune responses are raised at the site(s) of pathogen entry, as well as systemically.

Extended Release Systems:

A first extended release system includes matrix systems, in which the agent is embedded or dispersed in a matrix of another material that serves to retard the release of the agent into an aqueous environment (i.e., the luminal fluid of the GI tract). When the agent is dispersed in a matrix of this sort, release of the drug takes place principally from the surface of the matrix. Thus the drug is released from the surface of a device, which incorporates the matrix after it diffuses through the matrix or when the surface of the device erodes, exposing the drug. In some embodiments, both mechanisms can operate simultaneously. The matrix systems may be large, i.e., tablet sized (about 1 cm), or small (<0.3 cm). The system may be unitary (e.g., a bolus), may be divided by virtue of being composed of several sub-units (for example, several capsules which constitute a single dose) which are administered substantially simultaneously, or may comprise a plurality of particles, also denoted a multiparticulate. A multiparticulate can have numerous formulation applications. For example, a multiparticulate may be used as a powder for filling a capsule shell, or used per se for mixing with food to ease the intake.

In a specific embodiment, a matrix multiparticulate, comprises a plurality of the agent-containing particles, each particle comprising the agent and/or an analogue thereof e.g. in the form of a solid solution/dispersion with one or more excipients selected to form a matrix capable of controlling the dissolution rate of the agent into an aqueous medium. The matrix materials useful for this embodiment are generally hydrophobic materials such as waxes, some cellulose derivatives, or other hydrophobic polymers. If needed, the matrix materials may optionally be formulated with hydrophobic materials, which can be used as binders or as enhancers. Matrix materials useful for the manufacture of these dosage forms such as: ethylcellulose, waxes such as paraffin, modified vegetable oils, carnauba wax, hydrogenated castor oil, beeswax, and the like, as well as synthetic polymers such as poly(vinyl chloride), poly(vinyl acetate), copolymers of vinyl acetate and ethylene, polystyrene, and the like. Water soluble or hydrophilic binders or release modifying agents which can optionally be formulated into the matrix include hydrophilic polymers such as hydroxypropyl cellulose (HPC), hydroxypropyl methyl cellulose (HPMC), methyl cellulose, poly(N-vinyl-2-pyrrolidinone) (PVP), poly(ethylene oxide) (PEO), poly(vinyl alcohol) (PVA), xanthan gum, carrageenan, and other such natural and synthetic materials. In addition, materials, which function as release-modifying agents include water-soluble materials such as sugars or salts. Preferred water-soluble materials include lactose, sucrose, glucose, and mannitol, as well as hydrophilic polymers like e.g. HPC, HPMC, and PVP.

In a specific embodiment, a multiparticulate product is defined as being processed by controlled agglomeration. In this case the agent is dissolved or partly dissolved in a suitable meltable carrier and sprayed on carrier particles comprising the matrix substance.

Dose:

An effective dose of a composition of the presently disclosed subject matter is administered to a subject in need thereof. A “therapeutically effective amount” or a “therapeutic amount” is an amount of a therapeutic composition sufficient to produce a measurable response (e.g., a biologically or clinically relevant response in a subject being treated). The response can be measured in many ways, as discussed above, e.g. cytokine profiles, cell types, cell surface molecules, etc. Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

The compositions described above may be administered to animals including human beings in any suitable formulation. For example, compositions comprising live cells may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

The invention has been described in detail with reference to preferred embodiments thereof. However, it will he appreciated that those skilled in the art, upon consideration of this disclosure, may make modifications and improvements within the spirit and scope of the invention. The following non-limiting examples are illustrative of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### Combination Vaccine

The results obtained are shown below (Table 1), using a combination of cell based 293-gp96-Ig-SIV-gag, retanef (Rev-Tat-Nef), gp160 vaccination with recombinant gp120-protein, using gp96-Ig as adjuvant to protect macaques against rectal SIVmac251 challenge.

Vaccine:

Live, irradiated 293-SIVgag, retanef (Rev-Tat-Nef), gp160-gp96-Ig cells.

Dose:

Number of Cells secreting 10 μg gp96-Ig in 24 h.

Vaccination Schedule:

Week 0, 6, 26.

Vaccination Route:

intraperitoneal.

Three groups of 12 macaques, 1-2 females in each group, 2-3 MamuA1 in each group, Trim5α as indicated.

Group 1 vaccinated with 293-SIVgag, retanef gp160-gp96-Ig cells.

Group 2 vaccinated with 293-SIV gag, retanef: gp 160-gp96-Ig cells+100 μg recombinant gp120 protein through the same needle, gp96-Ig as adjuvant in trans.

Group 3 mock vaccinated with 293-gp96-Ig cells (no SIV antigens).

Challenge:

6 weeks after final vaccination.

Route:

Rectal instillation.

Virus:

SIV_{mac251} (swarm virus).

Dose: 120 TCID

Schedule: weekly rectal challenges×5 or until viral titer detected in plasma. The data show that 6 of 12 control, mock vaccinated macaques became infected after one rectal challenge. Only 1 of 12 macaques vaccinated with gp96-Ig-SIV alone and 0 of 12 macaques vaccinated with the gp96-Ig-SIV+gp120 protein combination became infected. The combination vaccination appears to provide the best protection from SIV infection.

TABLE 1

| Group 1 | | | Group 2 | | | Group 3 | | |
|---|---|---|---|---|---|---|---|---|
| Gp96.SIV-Ig | | | Gp96.SIV-Ig + gp120 | | | Mock Gp96-Ig | | |
| | Mamu, | | | Mamu, | | | Mamu, | |
| | Trim5α | Copies/ | | Trim5α | Copies/ | | Trim5α | Copies/ |
| # | Gender | ml | # | Gender | ml | # | Gender | ml |
| P417 | A1, B4, T+ | <50 | P421 | A1, T+ | <50 | P418 | A1, B1, 4, T+ | <50 |
| P419 | A1, B1 | <50 | P429 | A8, B4 | <50 | P420 | A1T+ | <50 |
| P422 | A2 | <50 | P439 | A2, 11, B1, 4 | <50 | P424 | A2, B4, T+ | 77845 |
| P423 | A2, T+ | <50 | P440 | A8, T+ | <50 | P425 | A2 | 484 |
| P426 | A11, B4, T+ | <50 | P442 | A1, B4 | <50 | P432 | A8, B4, T+ | 676 |
| P427 | | 222 | P430 | | <50 | P433 | A8, T+ | <50 |
| P428 | A8, B4 | <50 | P470 | F | <50 | P438 | B4 | <50 |
| P444 | A1, T+ | <50 | P475 | A8, B1, F | <50 | P441 | A1, 8, B1, 4 | <50 |
| P471 | F | <50 | P504 | A2, B1 | <50 | P473 | A2, F, T+ | <50 |
| P472 | A2, A8, F | <50 | P505 | | <50 | P474 | A8, F | 2098 |
| P510 | A2 | <50 | P506 | T+ | <50 | P508 | A2, B1, B8 | 1547 |
| P511 | A2 | <50 | P507 | A2, B8 | <50 | P509 | B1 | 37122 |

T⁺—Trim 5α high; F—Female. Data from 5 days after first rectal challenge.

Table 2 shows the comparison of Kaplan Meyer infection-curves between 293^{SIV}Gp96-Ig+Gp120 protein versus mock.

TABLE 2
Comparison of Survival Curves

| | |
|---|---|
| Log-rank(Mantel-Cox)Test | |
| Chi square | 7.863 |
| df | 1 |
| P value | 0.0050 |
| P value summary | ** |
| Are the survival curves significantly different? | Yes |
| Gehan-Breslow-Wilcoxon Test | |
| Chi square | 11.11 |
| df | 1 |
| P value | 0.0009 |
| P value summary | *** |
| Are the survival curves significantly different? | Yes |
| Median survival | |
| Mock | 1.500 |
| gp96 + gp120 | 2.500 |
| Ratio | 0.6000 |
| 95% Cl of ratio | 0.1786 to 1.021 |
| Hazard Ratio | |
| Ratio | 4.923 |
| 95% Cl of ratio | 0.616 to 15.00 |

### Example 2

### Vaccine-Cells Secreting gp96^{SIV}Ig Combined with gp120-Protein Protect from GP-22 Mucosal Infection with Highly Pathogenic SIV_{mac25}

The gp96-Ig was genetically engineered as a fusion protein by replacing the KDEL sequence of gp96 with Fc of IgG1 and secreted by cells containing the antigens of interest, to study the molecular and cellular mechanisms of CTL induction in animal models and as cancer vaccines in IRB/OBA/FDA approved clinical trials. Secreted gp96-Ig was a powerful adjuvant for MHC I cross presentation of gp96-chaperoned peptides and CTL priming and adjuvant for MHC II presentation of protein antigens and antibody production. The unique properties and immunogenicity of cell secreted gp96-Ig was used to evaluate it as protective vaccine against SIV_{mac251} infection.

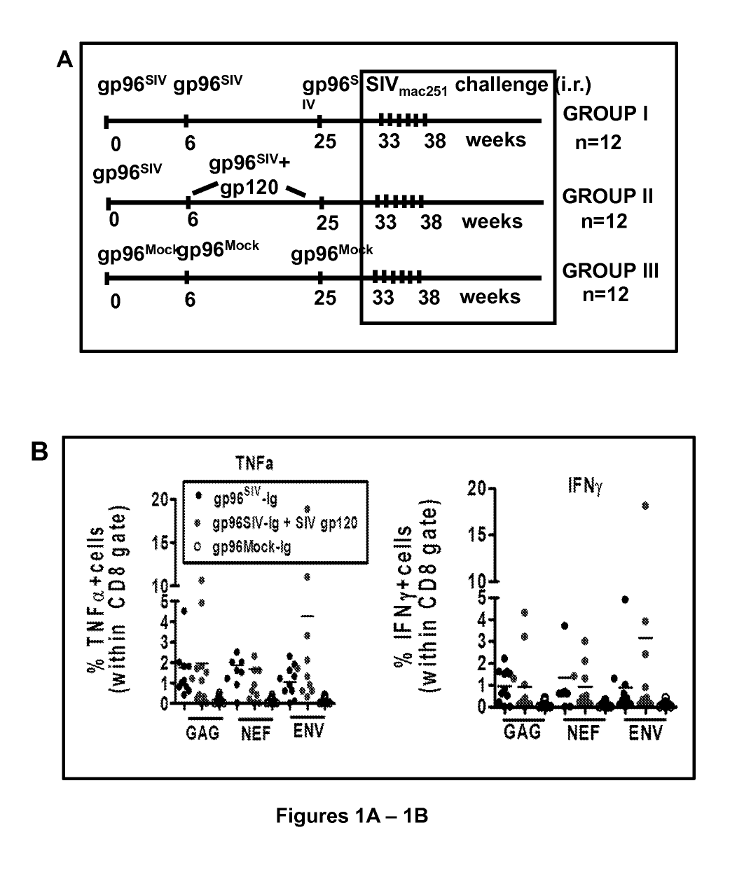

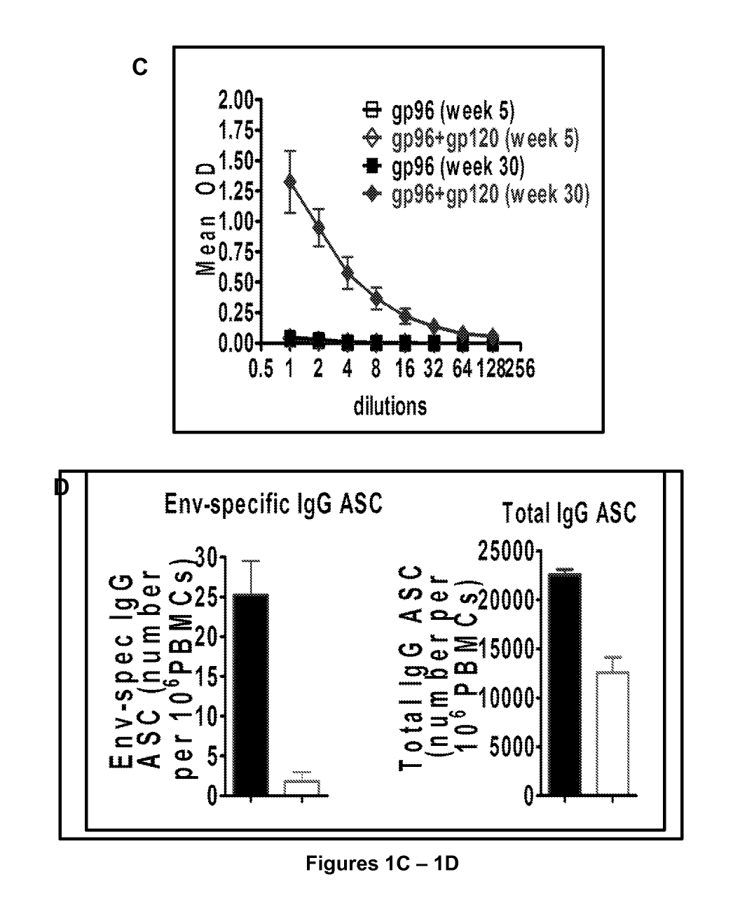

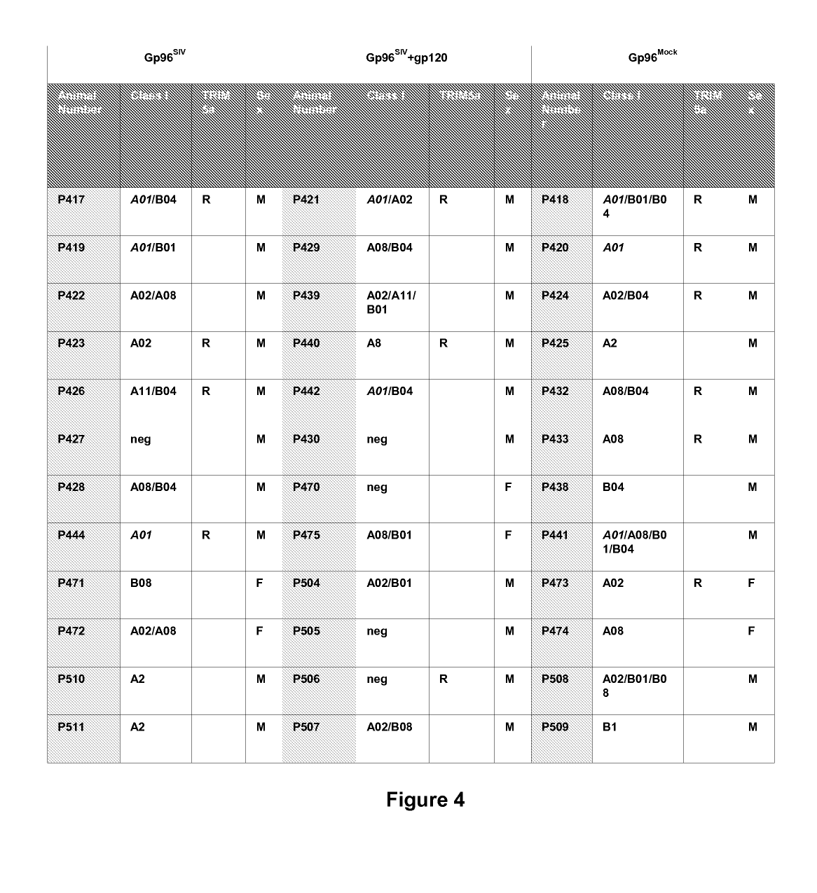

In initial immunogenicity and dose finding studies, it was found using intraperitoneal vaccination of 7 macaques with 293-gp96^{SIV}-Ig vaccination remarkable mucosal levels of polyepitope specific CTL for SIV gag, tat, nef and gp120 secreting IFN-γ and IL-2 upon stimulation. To determine the protective activity of the gp96^{SIV}Ig-vaccine approach, 36 Indian-origin rhesus macaques (Macaca mulatta) well balanced by gender, MHC type and TRIM5α expression (FIG. 4) were immunized by the intraperitoneal route in 3 groups of 12 macaques with (1) 293-gp96^{SIV}Ig cells secreting 10 μg gp96^{SIV}Ig per 24 h; (2) 293-gp96^{SIV}Ig+100 μg gp120-protein (ABL Inc, native protein from SIV_{mac251}); (3) 293-gp96-Ig (mock, no SIV antigens). The non-replicating (irradiated) vaccine cells are alive and secret gp96-Ig for 3-4 days. Macaques were primed i.p. at week 0 and boosted at week 6 and 25 (FIGS. 1A-1D). Macaques in group II received 100 μg native gp120 envelop protein (SIV_{mac251}) in HBSS at week 5 and 25 through the same needle as vaccine cells.

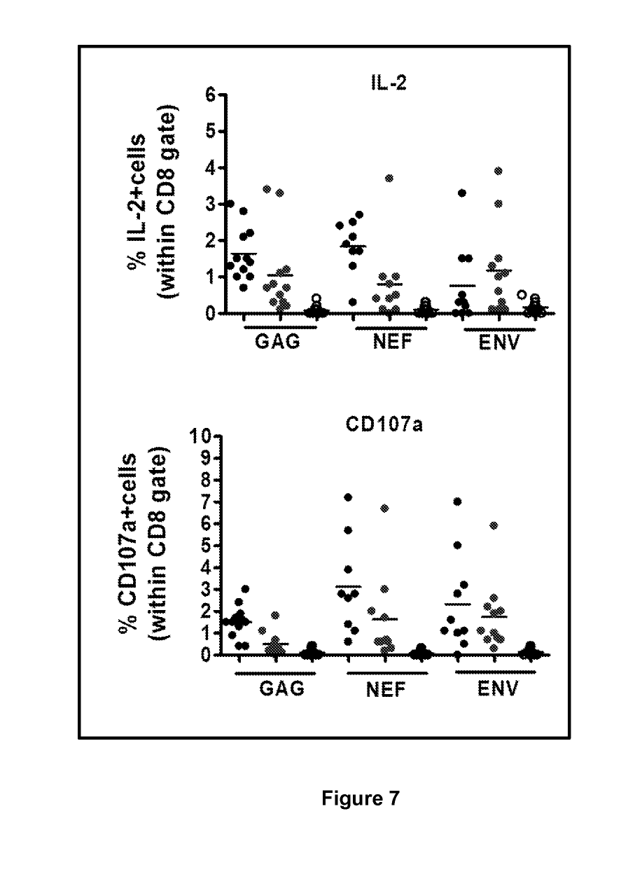

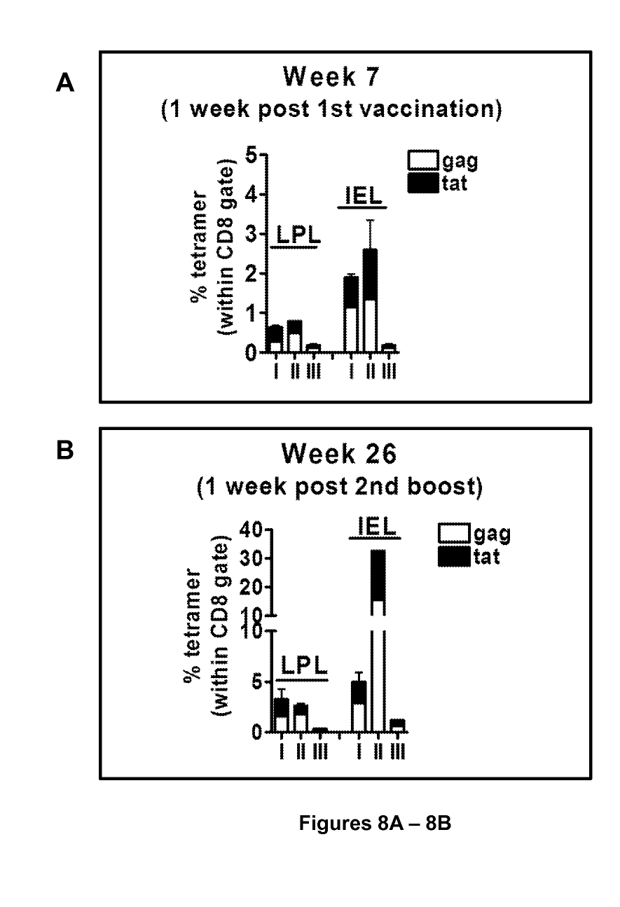

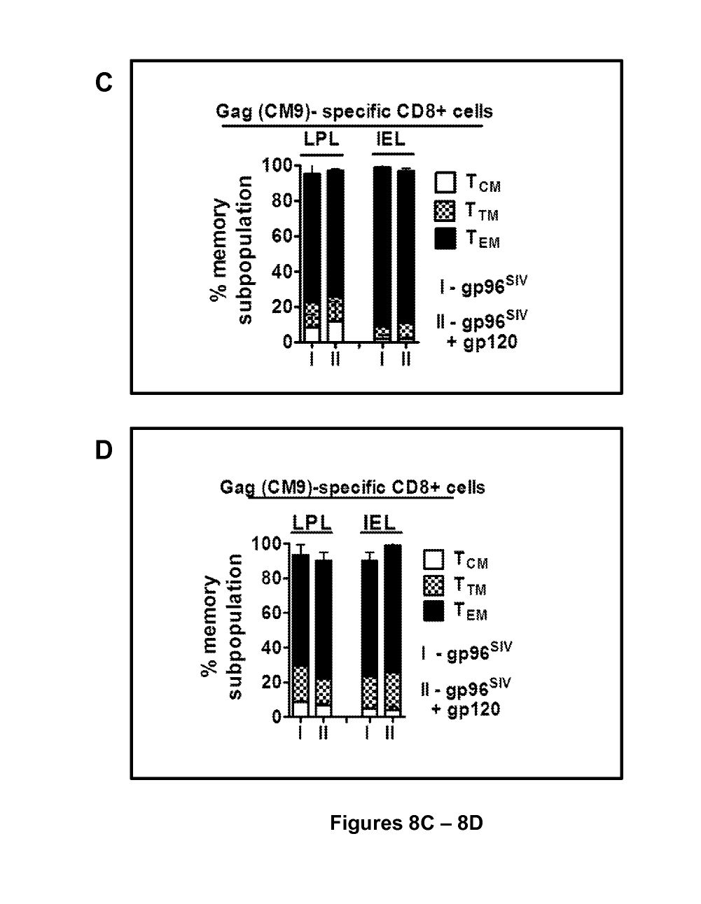

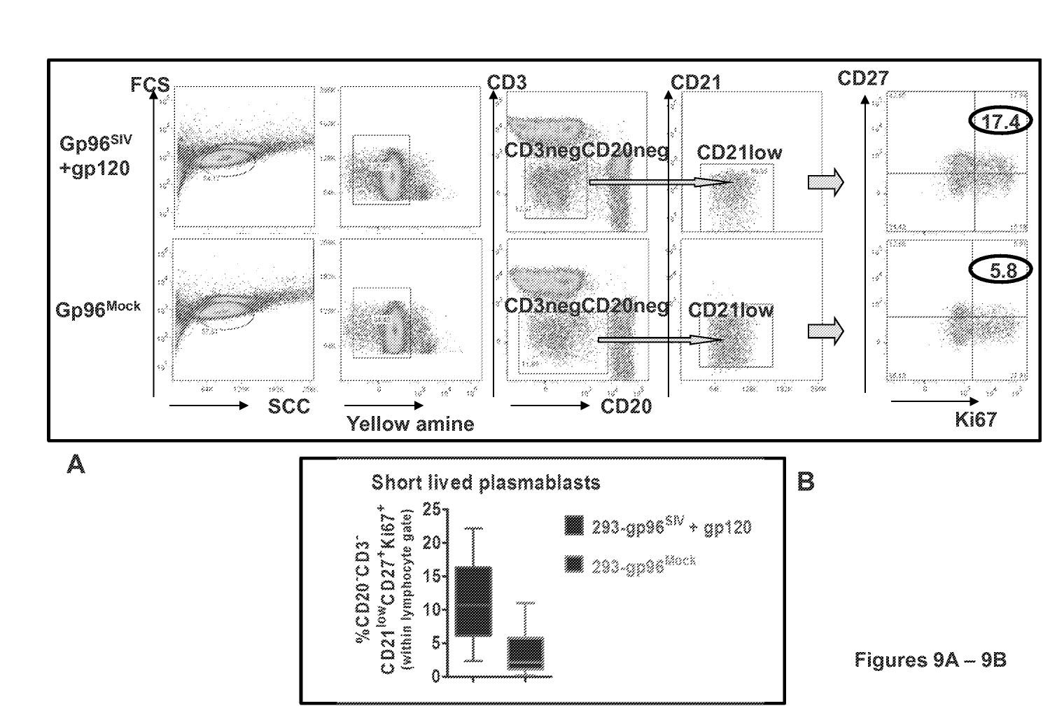

The immune response to 293-gp96SIVIg or mock vaccination was determined in week 7 and 26. Cellular immune responses were measured by multiparameter intracellular cytokine staining (ICS) assay and by SIV gag and tat tetramers (FIG. 1B and FIGS. 7, 8A-8D); humoral immune responses were measured by ELISA for gp120-specific antibodies (FIG. 1C) and by ELIspot assay for gp120-specific, antibody secreting cells (FIG. 1D) and by multiparameter staining for plasmablasts (FIGS. 9A, 9B). Immunological analysis one week after the last vaccination showed extremely powerful CTL activation in the intestinal mucosa in rectal LPL and IEL (FIG. 1B and FIG. 7). SIV gag and tat specific CTL responses were determined in 8 MAMU-A01+ Rhesus monkeys (FIGS. 8A, 8B). Both vaccine regimens (gp96^{SIV}Ig alone and the combination with gp120 protein) elicited a high level of Gag and Tat-specific immune responses in the lamina propria and especially in the intraepithelial compartment (FIG. 8B). The prime/boost gp96^{SIV}-Ig vaccine strategy in rhesus macaques resulted preferentially in the development of T_{EM} in the lamina propria and epithelial layer (FIGS. 8C, 8D) in agreement with data conducted in the inventors' laboratory.

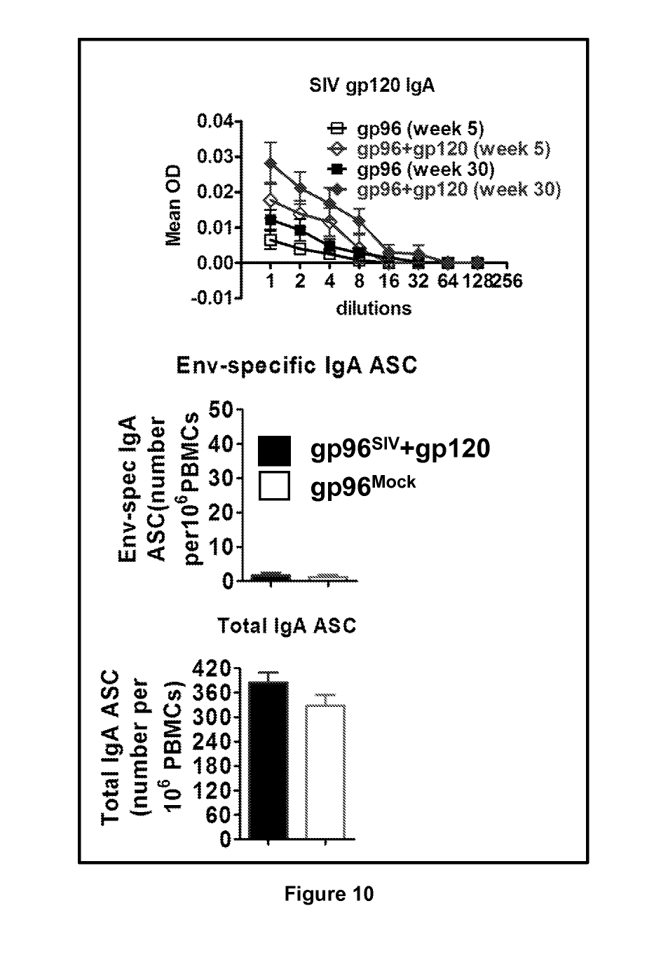

Addition of gp120 protein to 293-gp96^{SIV}Ig vaccination is required for antibody production (FIG. 1C). Significant SIV gp120-antibody titers and antibody secreting cells in blood are only detected when recombinant gp120 protein is mixed with the 293-gp96^{SIV}-Ig cells and coinjected. Gp120 specific antibodies in blood were all of IgG isotypes, except IgA (FIG. 10).

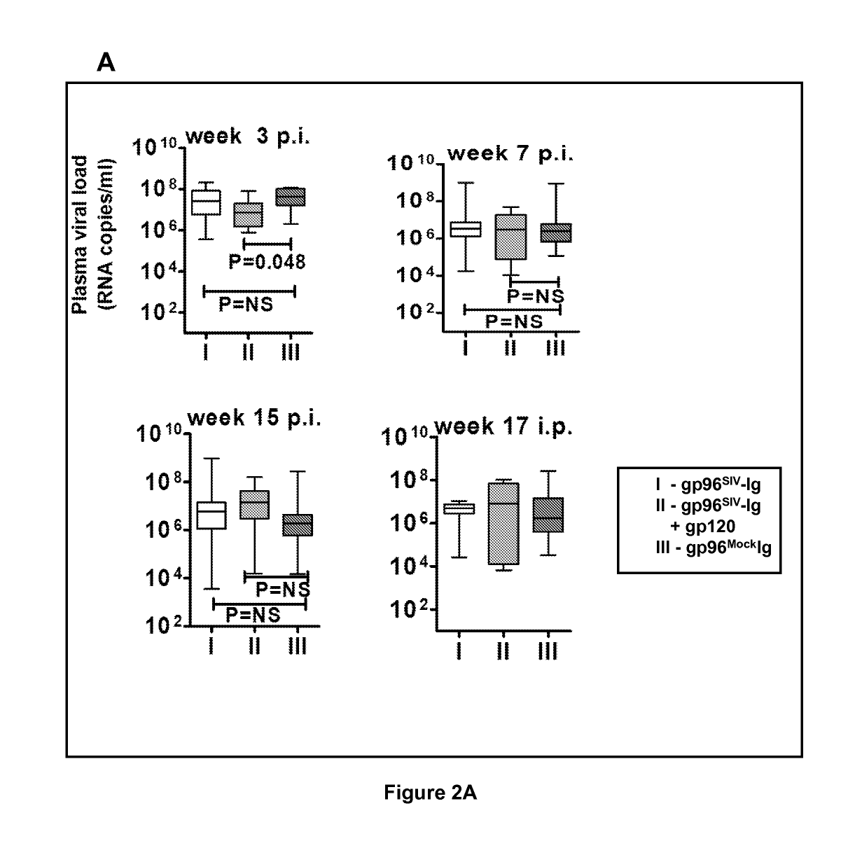

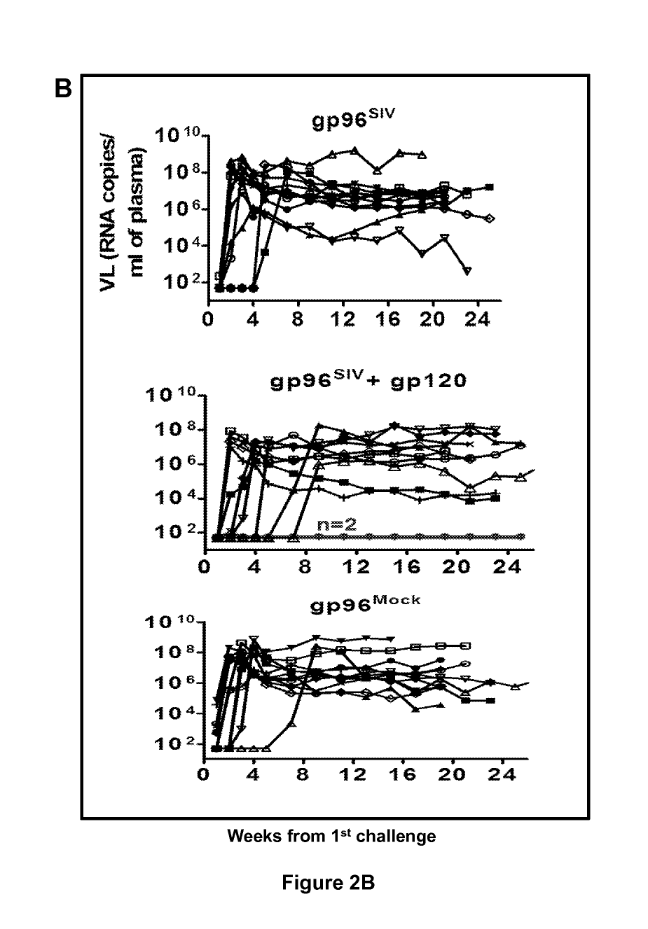

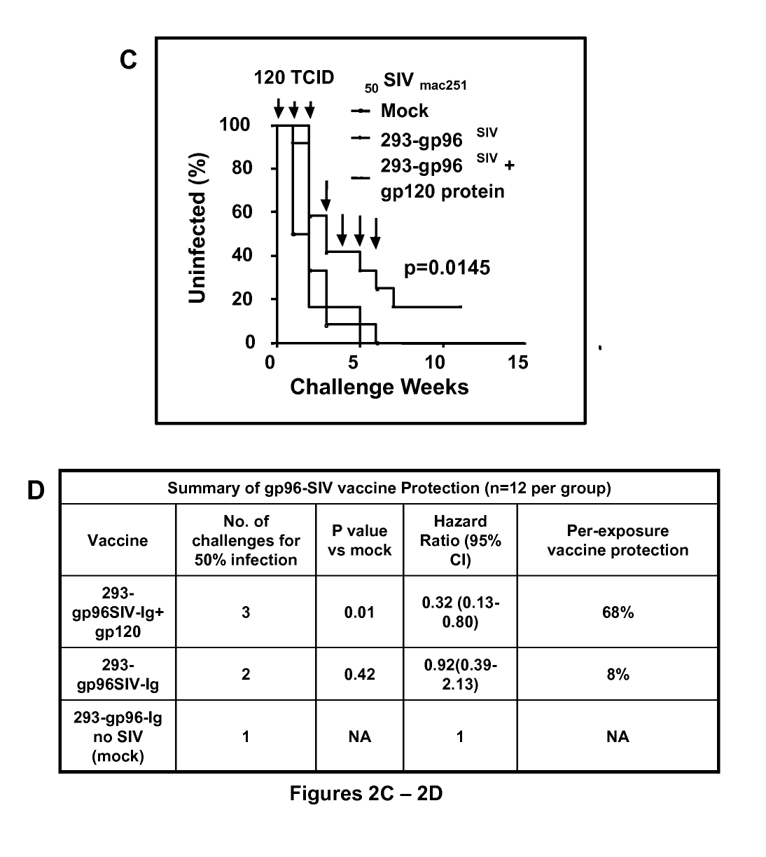

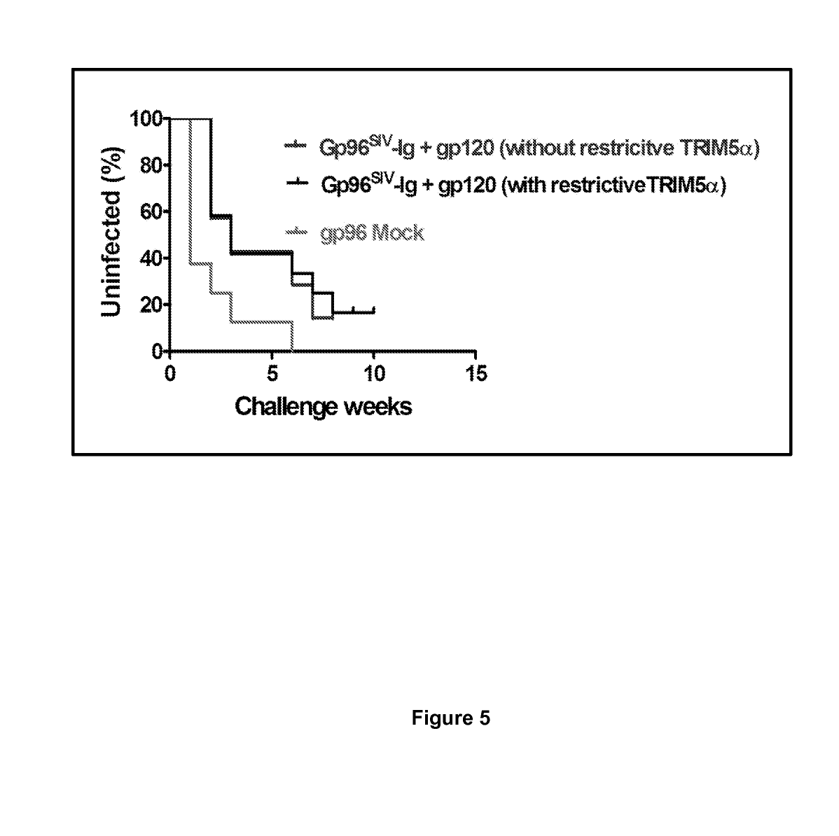

To evaluate the protective power of the immune response induced by gp96^{SIV}Ig vaccines, all 36 macaques were challenged starting at week 33 (5 weeks after the last vaccination) with seven weekly intrarectal inoculations of the SIV_{mac251} virus (120 TCID₅₀, NIH stock provided by Nancy Miller and Genoveffa Franchini) (FIG. 1A). Challenge of individual macaques was discontinued when they had positive virus titers, assessed 5 days after each challenge. Intrarectal inoculation of 120TCID₅₀ SIV_{mac251} generates 3-4 founder viruses in control, unvaccinated monkeys. It was found to be statistically significant (p=0.01; HR=0.27; 95% CI 0.09, 0.79, Gehan, Breslow test) vaccine protection of gp96^{SIV}Ig+gp120 vaccinated macaques against SIV acquisition after 7 rectal challenges (FIG. 2C) corresponding to a vaccine efficacy of 73% (VE=100×[1−HR]). Protection was completely unaffected by the presence of TRIM5α or restrictive MHC alleles (FIG. 5). After the first challenge, 50% of mock control macaques became infected, compared with only 4.1% of the gp96^{SIV}-Ig vaccinated animals. Macaques that received gp96^{SIV}Ig+gp120 required three challenges to infect 50% of animals (FIG. 2D). Two challenges were required for 50% infection for animals that received gp96^{SIV}Ig alone.

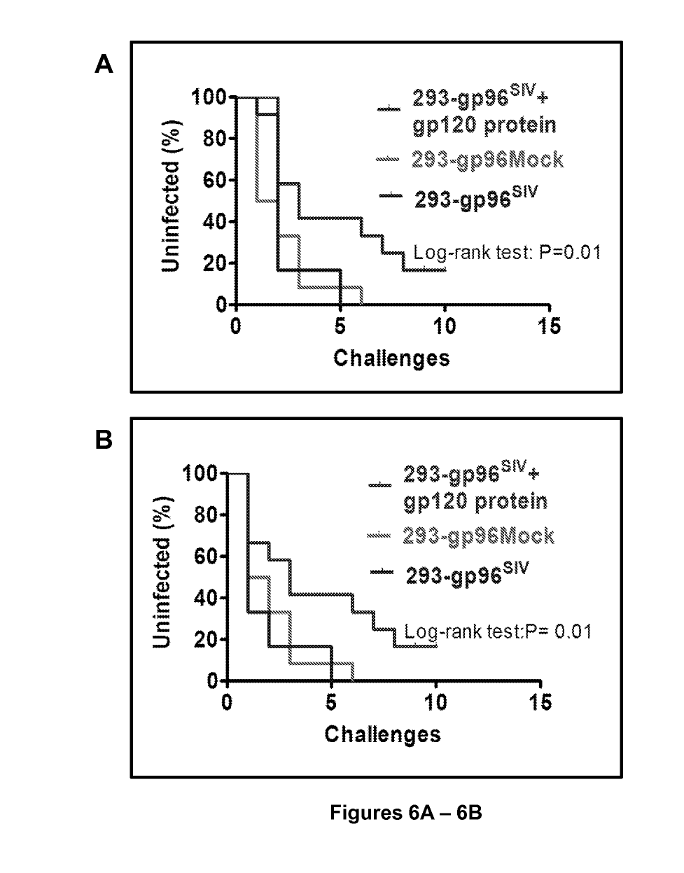

It was observed that some animals had very high virus titers on the day of first detection. For those with viral loads exceeding 10⁶ RNA copies/ml plasma it is possible that the animal had already been infected one week earlier but with as yet undetectable virus in blood. Rescoring data under this conservative assumption also yields significant protection (p=0.017; HR=0.39; 95% CI 0.17, 0.88) (FIGS. 6A, 6B).

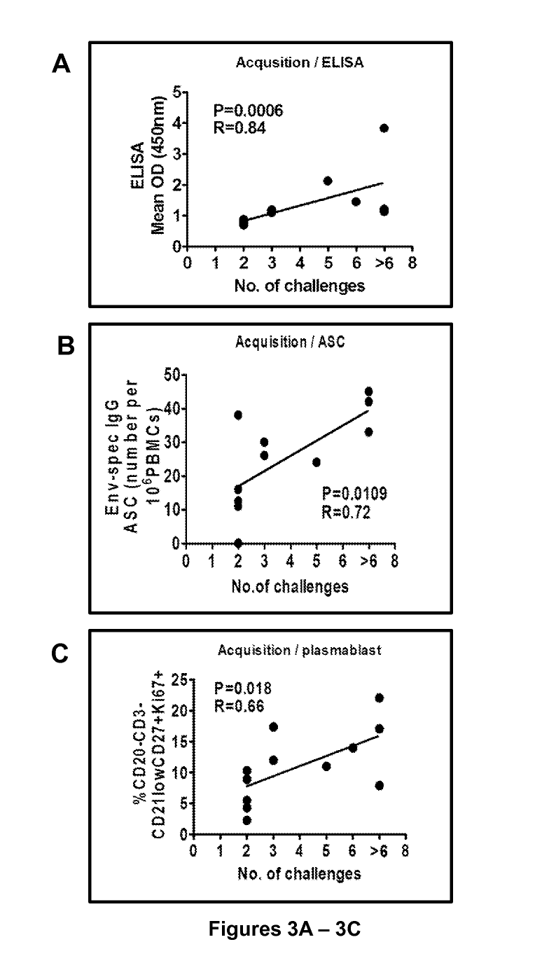

Gp96^{SIV} alone without added recombinant gp120 did not provide protection (FIGS. 2C, 2D). Likewise recombinant gp120 alone is not protective. Although infection did occur in most macaques vaccinated with the combined vaccine (FIG. 2B), viral acquisition required significantly more challenges than in the other groups (FIGS. 2B-2D), indicating a substantial degree of immunity. Gp120 protein in the vaccine cocktail was important for the generation of antibody (FIGS. 3A-3C), in addition to CD8 CTL generated by gp96-mediated MHC I cross presentation of SIV antigens.

Infected macaques showed peak viral loads on day 14 following infection (FIG. 2B) and then relatively stable mean set point viral loads of SIV RNA copies per ml. Macaques vaccinated gp96^{SIV}-Ig+gp120 had a 1 log reduction of the mean peak viral load compared with mock controls in week 3 (p=0.048, Wilcoxon rank-sum test). Overall, however, vaccinated groups did not show virological control once infected (FIG. 2A).

Next, it was evaluated whether immunological correlates of protection against acquisition of infection, defined as the number of challenges required to establish infection. Protection against acquisition of infection was best correlated with Env-specific ELISA antibody responses (FIG. 3A, P=0.0006, Spearman rank-correlation test) and frequency of Env-specific antibody secreting cells (FIG. 3B) and plasmablasts (FIG. 3C) prior to challenge. Cellular immune parameters (Gag, Nef and Env specific CD8⁺ T cell responses before challenge) were not significantly correlated with protection against acquisition of infection. Correlative analysis of vaccine-induced pre-challenge levels of cellular and humoral responses and plasma viral levels in the animals that become infected, demonstrated no significant correlation with T cell or B cell responses.

In summary, these data demonstrate for the first time that secreted heat shock protein gp96 can serve as the adjuvant for both CD8 CTL priming/expansion and for gp120 specific antibodies. Furthermore, combination of cell-based vaccination with recombinant protein can protect against acquisition of highly pathogenic, neutralization-resistant SIV_{mac251} challenges in rhesus monkeys. Despite the similarity of protection from SIV infection this vaccine approach is significantly different from that of other groups and offers flexibility including ease of combination with additional adjuvants to further improve vaccine efficacy. The cellular gp96-Ig vaccine approach is also being tested for different targets such as cancers (breast, non-small cell lung, bladder, ovarian cancer) and other infectious agents (HCV, malaria) to name some under investigation.

1-49. (canceled)

50. A method of preventing a primate lentiviral infection in a subject comprising administering to the subject a combination of (a) a pharmaceutical composition comprising at least a portion of gp120 from the primate lentivirus and a pharmaceutically acceptable carrier, and (b) a vaccine comprising a plurality of live irradiated host cells each co-expressing (i) at least three different primate lentivirus antigens selected from the group consisting of: at least a portion of Gag, at least a portion of Tat, at least a portion of Rev, a least a portion of Nef, and at least a portion of gp160, and (ii) a heat shock protein modified to be secreted from each of the host cells.

51. The method of claim 1, wherein the modified heat shock protein is gp96 lacking a functional endoplasmic reticulum retention sequence.

52. The method of claim 1, wherein the vaccine is mixed with the pharmaceutical composition prior to administration.

53. The method of claim 1, wherein the at least a portion of gp120 from the primate lentivirus is recombinant gp120.

54. A method of preventing a primate lentiviral infection in a subject previously administered a pharmaceutical composition comprising at least a portion of gp120 from the primate lentivirus and a pharmaceutically acceptable carrier, the method comprising administering to the subject a vaccine comprising a plurality of live irradiated host cells each co-expressing (i) at least three different primate lentivirus antigens selected from the group consisting of: at least a portion of Gag, at least a portion of Tat, at least a portion of Rev, a least a portion of Nef, and at least a portion of gp160, and (ii) a heat shock protein modified to be secreted from each of the host cells.

55. The method of claim 5, wherein the modified heat shock protein is gp96 lacking a functional endoplasmic reticulum retention sequence.

56. The method of claim 5, wherein the at least a portion of gp120 from the primate lentivirus is recombinant gp120.

57. A method of preventing a primate lentiviral infection in a subject previously administered a vaccine comprising a plurality of live irradiated host cells each co-expressing (i) at least three different primate lentivirus antigens selected from the group consisting of: at least a portion of Gag, at least a portion of Tat, at least a portion of Rev, a least a portion of Nef, and at least a portion of gp160, and (ii) a heat shock protein modified to be secreted from each of the host cells, the method comprising administering to the subject a pharmaceutical composition comprising at least a portion of gp120 from the primate lentivirus and a pharmaceutically acceptable carrier.

58. The method of claim 8, wherein the modified heat shock protein is gp96 lacking a functional endoplasmic reticulum retention sequence.

59. The method of claim 8, wherein the at least a portion of gp120 from the primate lentivirus is recombinant gp120.

60. A vaccine for preventing a primate lentiviral infection, the vaccine comprising (a) a plurality of live irradiated host cells each co-expressing (i) at least three different primate lentivirus antigens selected from the group consisting of: at least a portion of Gag, at least a portion of Tat, at least a portion of Rev, a least a portion of Nef, and at least a portion of gp160, and (ii) a heat shock protein modified to be secreted from each of the host cells, and (b) a pharmaceutical composition comprising at least a portion of gp120 from the primate lentivirus and a pharmaceutically acceptable carrier.

61. The vaccine of claim 11, wherein the modified heat shock protein is gp96 lacking a functional endoplasmic reticulum retention sequence.

62. The vaccine of claim 11, wherein the at least a portion of gp120 from the primate lentivirus is recombinant gp120.


SEQUENCE LISTING (2 sequences)

SEQ ID NO: 1 - protein, 8 aa, Artificial Sequence
  dendrimer sequence
CTPYINQM

SEQ ID NO: 2 - protein, 8 aa, Artificial Sequence
  dendrimer sequence
ITPESANL